(19) United States
(12) Patent Application Publication
(10) Pub. No.: US 2007/0196904 A1
(43) Pub. Date: Aug. 23, 2007

(54) Method for the production of chiral secondary alcohols

(76) Inventors: Thomas Dax, Frankenmarkt (AT); Michael Stanek, Linz (AT); Peter Poechlauer, Linz (AT)

Correspondence Address: NIXON & VANDERHYE, PC, 901 NORTH GLEBE ROAD, 11TH FLOOR, ARLINGTON, VA, 22203, US

(21) Appl. No.: 10/592,758

(22) PCT Filed: Feb. 25, 2005
(86) PCT No.: PCT/EP05/01977

§ 371 Date: Sep. 14, 2006

(30) Foreign Application Priority Data
Mar. 18, 2004 (AT) A 471/2004

(51) Int. Cl. C12P 13/00

(52) U.S. Cl. 435/128; 558/451

(57) ABSTRACT

Disclosed is a method for producing chiral, secondary alcohols of formula (I), wherein A represents an aromatic, heterocyclic, or alicyclic ring or a ring system with 4 to 20 C atoms, n represents 0, 1, 2, 3, 4, or 5, R represents halogen, OH, an O— protective group, NO₂, N,N—R2,R3 amine, R2 and R3 representing C1-C6 alkyl, phenyl, or benzyl, N,N—R2,R3-amino-C₁-C₆ alkyl, C₁-C₆ alkyl, C₁-C₆ haloalkyl, C₁-C₆ alkoxy, C₁-C₄ alkoxycarbonyl, or CN, and R1 represents N,N—R2,R3 amine, N,N—R2,R3-amino-C₁-C₆ alkyl, C₁-C₁₂ alkyl, C₁-C₆-haloalkyl, C₁-C₄ alkoxycarbonyl, C₁-C₆ alkoxy-C₁-C₆ alkyl, or a C₂-C₅ alkylene radical that forms a ring system along with the A radical. According to the inventive method, a) a ketone of formula (II) is optionally reduced to the corresponding racemic alcohol of formula (III) by means of an aliphatic C₁-C₆ alcohol in the presence of a transition metal catalyst and a base, and b) the alcohol of formula (III) is reacted to a mixture of (R) ester of formula (IV), wherein R4 represents H or C₁-C₅ alkyl, and (S) alcohol of formula (I) in the presence of an esterification catalyst and an acyl donor, whereupon the (S) alcohol is isolated from the reaction mixture by means of crystallization or distillation according to the aggregate state thereof.

The present invention relates to a method for the production of chiral, secondary alcohols in an optical purity up to 100% ee and in up to 100% yield.

Chiral, secondary alcohols such as, for instance, (S)-1-phenylethanol derivatives are valuable intermediates for the production of pharmaceuticals and agrochemicals.

The production of chiral, secondary alcohols such as, for instance, (S)-1-(3,5-bis(trifluoromethyl)phenyl)-ethan-1-ol is disclosed, for example, by Chemical Abstracts No. 136:68817, according to which the desired compound is obtained by asymmetric reduction of bis-3,5-(trifluoromethyl)phenyl methyl ketone in the presence of dried microbial powder, NAD⁺ and/or NADP⁺ and a secondary alcohol, in a yield of only 52%. According to Organic Letters 3 (25), 4111-4113 (2001), various ketones are converted by hydrosilylation in the presence of a copper(II) fluoride/chiral diphosphine system as catalyst into the corresponding alcohols with moderate to high enantioselectivity (ee up to 92%). However, (S)-1-(3,5-bis(trifluoromethyl)phenyl)ethan-1-ol is only obtained in 88% yield with an ee of 85%.

WO 03043575 discloses a method for the production of (R)-1-(3,5-bis(trifluoromethyl)phenyl)ethan-1-ol in which acetophenone derivatives can be converted into optically pure (R)-1-phenylethanols or esters thereof in a racemization-esterification cycle (DKR—dynamic kinetic resolution).

It is object of the present invention to provide a novel method for the production of chiral, secondary alcohols, such as for instance (S)-1-phenylethanols, or substituted derivatives, which makes it possible to produce the desired compounds in higher optical purities compared with the prior art up to 100% ee and in higher yields of up to 100%, in a simple manner.

Unexpectedly, this object has been able to be achieved by, in a one-pot reaction, a racemate of an alcohol, which is produced if appropriate in advance from the corresponding ketone, being enzymatically esterified and, from the resultant mixture of S-alcohol and R-ester, the S-alcohol being able to be selectively crystallized out or distilled off.

The present invention therefore relates to a method for the production of chiral, secondary alcohols of the formula 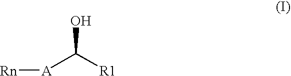
where A is an aromatic, heterocyclic ring, with O as heteroatom, or alicyclic ring or a ring system having 4 to 20 carbon atoms, n can be 0, 1, 2, 3, 4 or 5, and R is halogen, OH, O-protecting group, NO₂, N,N—R2,R3-amine, where R2 and R3 are equal to C₁-C₆-alkyl, phenyl or benzyl, N,N—R2,R3-amino-C₁-C₆-alkyl, where R2, R3 are as defined above, C₁-C₆-alkyl, C₁-C₆-haloalkyl, C₁-C₆-alkoxy, C₁-C₄-alkoxycarbonyl, or CN, and R1 is N,N—R2,R3-amine, where R2 and R3 are equal to C₁-C₆-alkyl, phenyl or benzyl, N,N—R2,R3-amino-C₁-C₆-alkyl, R2, R3 being as defined above, C₁-C₁₂-alkyl, C₁-C₆-haloalkyl, C₁-C₄-alkoxycarbonyl, or C₁-C₆-alkoxy-C₁-C₆-alkyl, or R1 is a C₂-C₅-alkylene radical which, together with the A radical, forms a ring system, a carbon atom in the alkylene chain optionally being replaced by an O atom, which comprises
a) if appropriate a ketone of the formula 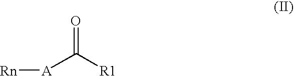
   where A, n, R and R1 are as defined above, being reduced by means of an aliphatic C₁-C₆-alcohol in the presence of a transition metal catalyst and a base to give the conjugate racemic alcohol of the formula 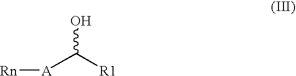
   where A, n, R and R1 are as defined above, and
b) the alcohol of the formula (III) being reacted in the presence of an esterification catalyst and an acyl donor from the group of C₂-C₆-alkenyl esters of aliphatic C₁-C₆-carboxylic acids to give a mixture of (R)-esters of the formula 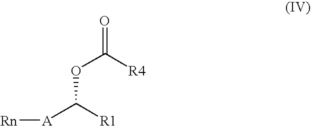
   where A, n, R and R1 are as defined above and R4 is H or C₁-C₅-alkyl, and (S)-alcohol of the formula (I), whereupon the (S)-alcohol, depending on its physical state, is isolated from the reaction mixture by crystallization or distillation.

In the inventive method, (S)-alcohols of the formula (I) are produced.

In the formula (I), A is an aromatic ring, heterocyclic ring with O as heteroatom, or alicyclic ring or a ring system having 4 to 20 carbon atoms.

Examples of these are phenyl, naphthyl, indenyl, cyclohexyl, decalinyl, tetralinyl, pyranyl, chromanyl etc.

n can be 0, 1, 2, 3, 4 or 5 and R halogen, such as fluorine, chlorine or bromine, OH, O-protecting group, NO₂, N,N—R2,R3-amine, with R2 and R3 identical to C₁-C₆-alkyl, phenyl or benzyl, N,N—R2,R3-amino-C₁-C₆-alkyl, R2, R3 being as defined above, C₁-C₆-alkyl, C₁-C₆-haloalkyl, C₁-C₆-alkoxy, C₁-C₄-alkoxycarbonyl, or CN.

C₁-C₆-Alkyl and C₁-C₆-alkoxy are taken to mean linear or branched alkyl or alkoxy radicals such as, for instance, methyl, methoxy, ethyl, ethoxy, isopropyl, isopropoxy, tert-butyl, n-butoxy, n-pentyl, n-hexyl etc.

Preference is given to C₁-C₄-alkyl and alkoxy radicals, particular preference to C₁-C₂-alkyl and alkoxy radicals.

C₁-C₆-Haloalkyl radicals are alkyl radicals having 1 to 6 carbon atoms which are substituted by 1 to 3 halogen atoms, such as fluorine, chlorine or bromine, such as for instance trifluoromethyl, trifluoroethyl, etc.

Preferred haloalkyl radicals are C₁-C₄-alkyl radicals which are substituted by 1-3 fluorine or chlorine atoms, particularly preferably C₁-C₂-alkyl radicals which are substituted by 3 fluorine atoms.

C₁-C₄-Alkoxycarbonyl radicals are carboxylic ester radicals having 1 to 4 carbon atoms in the ester moiety.

As protecting group for the O atom, conventional O-protecting groups come into consideration, such as, for instance, methoxymethyl, tetrahydro-2-pyranyl, tetrahydro-2-furanyl, 1-ethoxyethyl, 1-methyl-1-methoxy-ethyl, tert-butyl, benzyl, trimethylsilyl, 4-chloro-phenyl, 4-nitrophenyl, etc.

N,N—R2,R3-amine, where R2 and R3 are identical to C₁-C₆-alkyl, phenyl or benzyl, are secondary amines such as, for instance, N,N-dimethylamine, N,N-dibenzylamine, N,N-diethylamine, etc.

Examples of N,N—R2,R3-amino-C₁-C₆-alkyl are N,N-dimethylaminomethyl, N,N-diethylaminomethyl, N,N-dimethylaminoethyl, etc.

The A radical of the compound of the formula (I) can be unsubstituted (n=0) or 1 to 5-fold substituted by R. If A is phenyl or cyclohexyl, it is preferably 1 to 4-fold substituted in positions 3, 4 and/or 2, particularly preferably 1 or 2-fold substituted, the substituents preferably both being situated at the 3 positions, or one of the radicals at the 4 position and the other at the 3 position.

R1 in the formula (I) is N,N—R2,R3-amine, where R2 and R3 are identical to C₁-C₆-alkyl, phenyl or benzyl, N,N—R2,R3-amino-C₁-C₆-alkyl, R2, R3 being as defined above, C₁-C₁₂-alkyl, C₁-C₆-haloalkyl, C₁-C₄-alkoxycarbonyl, or C₁-C₆-alkoxy-C₁-C₄-alkyl.

Preferably, R1 is C₁-C₆-alkyl, C₁-C₂-haloalkyl, or C₁-C₂-alkoxy-C₁-C₄-alkyl, particularly preferably C₁-C₄-alkyl.

R1 can also be a C₂-C₅-alkylene radical which, together with the A radical, forms a ring system, if appropriate a carbon atom being able to be replaced in the alkylene chain by an O atom. Examples of such ring systems are, for instance, tetralin, indane, chromane, etc.

Compounds of the formula (I) which can be produced according to the invention are correspondingly, for example, (S)-1-(3,5-bis(trifluoromethyl)phenyl)ethan-1-ol, (S)-1-(3,5-bis(trifluoromethyl)phenyl)propan-1-ol, (S)-1-(3,4-bis(trifluoromethyl)phenyl)ethan-1-ol, (S)-1-(p-fluoro)phenylethan-1-ol (S)-1,2,3,4-tetrahydro-1-naphthol, etc.

In the inventive method, as starting material, if appropriate use is made of a ketone of the general formula (II) which is reduced in the course of the method to give the corresponding racemic alcohol, or use is already made of the racemate of a chiral alcohol of the general formula (III).

The inventive method which is carried out as a one-pot reaction can be divided into 3 stages:

a) Reduction

In this stage a ketone of the general formula (II), in which the substituents A, n, R and R1 are as defined above, is reacted in the presence of a transition metal catalyst by means of an aliphatic C₁-C₆-alcohol to give a racemic alcohol of the general formula (III), in which the substituents A, n, R and R1 are as defined above.

A suitable C₁-C₆-alcohol is, for example, methanol, ethanol, n-propanol, isopropanol, etc.

Preference is given to a C₂-C₄-alcohol, particular preference to isopropanol.

Suitable transition metal catalysts are catalysts or catalyst complexes which are based on a transition metal compound, for instance as described in WO 03/043575.

According to WO 03/043575 these are compounds of the formula MₙXₚS_{q}Lᵣ, where
n is an even number from 1 to 4,
p, q and r independently of one another can be 0, 1, 2, 3 or 4,
M is a transition metal from the group Fe, Co, Ni, Re, Ru, Rh, Ir, Os, Pd, Pt or Sm, or mixtures thereof,
X is an anion from the group hydride, halide, carboxylate, alkoxy, hydroxyl or tetrafluoroborate,
S is a spectator ligand, a neutral ligand which is difficult to exchange, and is selected from the group of olefins, dienes, or the aromatics, such as benzene, toluene, xylene, cumene, cymene, naphthalene, anisole, chlorobenzene, indole, cyclopentadiene derivatives, tetraphenyl, cyclopentadienone, dihydroindole, tetrahydronaphthalene, gallic acid, benzoic acid and phenylglycine,
and L is a neutral ligand which is relatively easy to exchange for another ligand and is selected from the group of acetonitrile, DMSO, methanol, water, THF, DMF, pyridine and N-methylpyrrolidone.

The transition metal compound can also be converted into a transition metal complex by exchanging the neutral ligand for another ligand, or by complexing the transition metal compound with a ligand. Suitable ligands for complexing are likewise disclosed by WO 03/043575.

These are, for example, primary or secondary amines, alcohols, diols, aminoalcohols, diamine amino acids, amino acid amides, etc.

Preferably for the inventive method, use is made of catalysts which are based on Pd, Ru, Ir or Rh, particularly preferably Ru.

As complex-forming ligand, use is preferably made of racemic or optically pure amino acid amides.

For activation of the catalyst, a base is added. The base can be selected from the group of the alkali or alkaline earth metal carbonates or hydrogencarbonates. Examples of these are sodium carbonate, potassium carbonate, potassium hydrogencarbonate, sodium hydrogencarbonate, etc.

The catalyst is used in an amount of 0.005 to 0.1 mol %, based on ketone or racemic alcohol, preferably 0.01 to 0.03 mol %.

The reaction temperature is 65-90° C., preferably 70° C.-85° C.

The reaction time is 0.5 to 5 h, preferably 1 to 2 h.

b) Enzymatic Esterification

After reduction of the ketone is complete, excess alcohol is distilled off and an esterification catalyst and an acyl donor are charged at a temperature of 20-80° C., preferably 50-70° C.

As acyl donor, use can be made of C₂-C₆-alkenyl esters of aliphatic C₁ to C₆-carboxylic acids, preferably 1-propenyl, vinyl or isopropenyl propionate, or butyrate. The acyl donor which is used in an amount of 0.5 to 1.0 equivalents, based on the ketone, can be charged in its whole quantity, or added in a slowly metered manner.

As esterification catalysts, suitable enzymes are again those listed in WO 03/043575, for instance those having lipase activity or those having amidase and lipase activity which originate, for example, from Pseudomonas, Bacillus, Candida, etc.

Preferably, for the inventive method, use is made of immobilized lipases such as Novozym435® (Candida antarctica) or enzymes of the Amano PS type (Pseudomonas lipases), particularly preferably Novozym435®.

During the enzymatic esterification, preferably a vacuum of 200 to 700 mbar is applied in order to remove any volatile compounds such as acetone or acetaldehyde from the reaction mixture. The reaction time for the esterification is 2 to 6 hours at a reaction temperature of 50 to 80° C., preferably 65-75° C.

After the esterification, an (R) ester of the general formula (IV) and the (S)-alcohol of the general formula (I) are present in a molar ratio of about 1:1. Excess acyl donor is distilled off using an organic solvent, preferably using an aliphatic or aromatic hydrocarbon, for example toluene, benzene, heptane, cyclohexane or methylcyclohexane, and the reaction mixture diluted with the same solvent.

c) Product Isolation

For the workup, preferably a small amount of activated carbon and a filter aid are added to the reaction mixture and stirred for some time. The mixture is filtered off, the filtercake washed with solvent and the filtrate diluted with solvent. It is cooled, seeded and the product crystallized at temperatures of −20 to 0° C. The resultant (S)-alcohol of the general formula (I) is washed with solvent and dried at room temperature under vacuum.

In the case of a liquid, the (S)-alcohol is separated off from the (R)-ester by distillation.

In a preferred embodiment, the mother liquor or the distillation bottom phase is recycled.

For this, after separating off the product (by crystallization or distillation), the residue is freed from the solvent and charged back into the reactor. By adding methanol or ethanol, preferably methanol, and the base used for activating the catalyst, preferably potassium carbonate, the ester of the formula (IV) is cleaved into R-alcohol and methyl ester of a carboxylic acid, for example methyl propionate. Missing material can be added in the form of ketone (II) or racemic alcohol (III). The reaction is continued as described under a). The mother liquor can be recycled without losing purity and/or optical purity of the end product. By continued recycling of the crystallization mother liquor, substrates are 100% reacted.

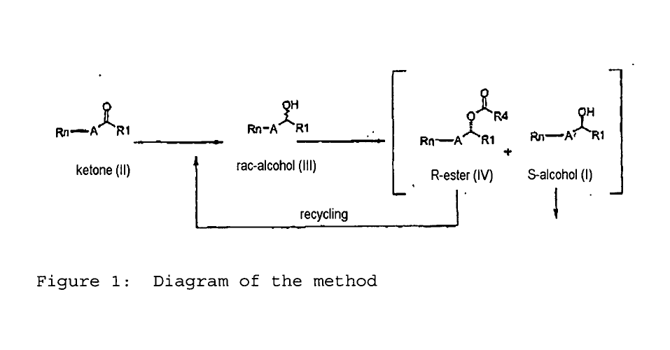

The process diagram is shown in FIG. 1.

By means of the inventive method, the corresponding (S)-alcohols are obtained in yields of up to 100% having an ee likewise of up to 100% in a simple one-pot method.

### EXAMPLE 1

Into a reactor rendered inert, there are charged, one after the other, 150 g (0.586 mol) of 1,3-bis(trifluoromethyl)acetophenone, 34 mg (0.056 mmol) of [RuCl₂(p-cymene)]₂, 19 mg (0.117 mmol) of alpha-phenyl-alpha-methylglycinamide, 261 mg (1.892 mmol) of potassium carbonate and 77.5 g of isopropanol. With stirring, the mixture is heated to 85° C. and kept for 1 hour at this temperature. Thereafter, vacuum is applied slowly and solvent distilled off to an endpoint of 100 mbar and 70° C. Subsequently, 3.9 g of Novozym435® are charged and, after applying 300 mbar vacuum, 46.9 g (0.468 mol) of vinyl propionate are slowly added and the mixture is stirred for a further 4 hours at 70° C. and 300 mbar. Thereafter, 60 ml of heptane are charged and distilled off to 100 mbar at 70° C. A suspension of 2 g of activated carbon and 0.5 g of Arbothix PE 100 in 60 ml of heptane are charged, the mixture is stirred for 10 min and the reaction mixture filtered hot. The filtercake was rinsed with 50 ml of heptane, all filtrates were combined and diluted with heptane. With stirring, the mixture was cooled to room temperature, seeded and cooled to −10° C. It was further stirred for 4 hours, the crystalline product was filtered over a vacuum filter and rinsed using 150 ml of cold heptane. The moist product was dried in vacuum at room temperature.

This produced 49 g (32.4%) of 1(S)-[3,5-bis(trifluoromethyl)phenyl]ethanol having an optical purity >99.9% ee and a purity of 99.9% GC.

### EXAMPLE 2

### Recycling Mother Liquor

Mother liquor from example 1 (101.4 g (0.396 mol) of recyclable material, stated as 1,3-bis(trifluoromethyl)acetophenone) was charged into a reactor, without fortification with fresh material, and heptane distilled off in vacuum. Thereafter, 1 g (7 mmol) of potassium carbonate and 100 ml of methanol were charged and the mixture heated to 75° C. It was refluxed for 1 h and thereupon methanol and methyl propionate were distilled off at atmospheric pressure.

34 mg (0.056 mmol) of [RuCl₂(p-cymene)]₂ and 19 mg (0.117 mmol) of alpha-phenyl-alpha-methylglycinamide were added together with 5 ml of isopropanol and 2 ml of methanol and heated under a vacuum of 500 mbar to 80° C. and kept at this temperature for 1 h. Thereafter, 50 ml of toluene were charged and distilled off slowly to a vacuum of 100 mbar. 35.2 g of vinyl propionate (0.351 mol) and 3 g of Novozym435® were added and the reaction mixture was stirred for 2 h at 70° C. 50 ml of heptane were charged and slowly distilled off to a vacuum of 200 mbar. Thereafter, a suspension of 2 g of activated carbon and 0.5 g of Arbothix® PE 100 in 100 ml of heptane was charged, stirred for 10 min and filtered off through a glass fiber filter. The filtercake was rinsed using 50 ml of heptane and the collected filtrates were cooled, seeded, and crystallized at a temperature of −20° C. The crystalline product was filtered, rinsed with cold heptane and dried in vacuum.

This produced 32 g of 1(S)-[3,5-bis(trifluoromethyl)-phenyl]ethanol (31.5% based on mother liquor used or 21.3% based on the amount of 1,3-bis(trifluoromethyl)acetophenone used in example 1) in a purity of 99.6 GC % and an optical purity >99.9% ee.

The mother liquor was able to be recycled without losing purity and/or optical purity of the end product. By continued recycling of the crystallization mother liquor, substrates were 100% reacted.

1. A method for the production of chiral, secondary alcohols of the formula 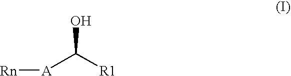
where A is an aromatic, heterocyclic ring, with O as heteroatom, or alicyclic ring or a ring system having 4 to 20 carbon atoms, n can be 0, 1, 2, 3, 4 or 5, and R is halogen, OH, O-protecting group, NO₂, N,N—R2,R3-amine, where R2 and R3 are equal to C₁-C₆-alkyl, phenyl or benzyl, N,N—R2,R3-amino-C₁-C₆-alkyl, where R2, R3 are as defined above, C₁-C₆-alkyl, C₁-C₆-haloalkyl, C₁-C₆-alkoxy, C₁-C₄-alkoxycarbonyl, or CN, and R1 is N,N—R2,R3-amine, where R2 and R3 are equal to C₁-C₆-alkyl, phenyl or benzyl, N,N—R2,R3-amino-C₁-C₆-alkyl, R2, R3 being as defined above, C₁-C₁₂-alkyl, C₁-C₆-haloalkyl, C₁-C₄-alkoxycarbonyl, or C₁-C₆-alkoxy-C₁-C₆-alkyl, or R1 is a C₂-C₅-alkylene radical which, together with the A radical, forms a ring system, a carbon atom in the alkylene chain optionally being replaced by an O atom, which comprises
a) if appropriate a ketone of the formula 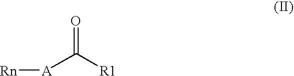
where A, n, R and R1 are as defined above, being reduced by means of an aliphatic C₁-C₆-alcohol in the presence of a transition metal catalyst and a base to give the conjugate racemic alcohol of the formula 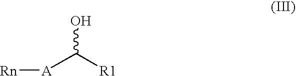
where A, n, R and R1 are as defined above, and
b) the alcohol of the formula (III) being reacted in the presence of an esterification catalyst and an acyl donor from the group of C₂-C₆-alkenyl esters of aliphatic C₁-C₆-carboxylic acids to give a mixture of (R)-esters of the formula 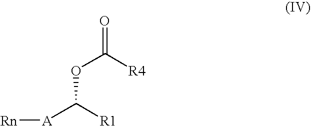
where A, n, R and R1 are as defined above and R4 is H or C₁-C₅-alkyl, and (S)-alcohol of the formula (I), whereupon the (S)-alcohol, depending on its physical state, is isolated from the reaction mixture by crystallization or distillation.

2. The method as claimed in claim 1, characterized in that, subsequent to the separation of the (S)-alcohol of the formula (I) by crystallization or distillation, the residue is recycled by freeing it from the solvent and charging it back into the reactor at stage a) or b).

3. The method as claimed in claim 1, characterized in that, in step a), use is made of transition metal catalysts based on Fe, Fe, Co, Ni, Re, Ru, Rh, Ir, Os, Pd, Pt or Sm, or mixtures thereof, or complexes with a ligand from the group of primary or secondary amines, alcohols, diols, aminoalcohols, diamines, amino acids, or amino acid amides, in the presence of a base from the group of the alkali metal or alkaline earth metal carbonates or hydrogencarbonates.

4. The method as claimed in claim 1, characterized in that, in step a), the reaction temperature is 65° C. to 90° C.

5. The method as claimed in claim 1, characterized in that, in step b), as esterification catalyst, use is made of enzymes having lipase activity or having amidase and lipase activity from the group Pseudomonas, Bacillus and Candida.

6. The method as claimed in claim 1, characterized in that, in step b), as acyl donor, use is made of vinyl propionate, vinyl butyrate, isopropenyl propionate or isopropenyl butyrate.

7. The method as claimed in claim 1, characterized in that step b) is carried out at a vacuum of 200 to 700 mbar.

8. The method as claimed in claim 1, characterized in that the reaction temperature for step b) is 50 to 80° C.
